(19) United States
(12) Patent Application Publication
(10) Pub. No.: US 2009/0093630 A1
(43) Pub. Date: Apr. 9, 2009

(54) CHIRAL SYNTHESIS OF DIAZEPINOQUINOLINES

(75) Inventors: Sreenivasulu Megati, New City, NY (US); Shilpa Bhansali, Wynnewood, PA (US); Christoph Dehnhardt, New York, NY (US); Subodh Deshmukh, White Plains, NY (US); Peter Fung, Northport, NY (US); Michael MacEwan, Monroe, NY (US); Robert J. Tinder, New City, NY (US)

Correspondence Address: CHOATE, HALL & STEWART LLP/WYETH, PATENT GROUP, TWO INTERNATIONAL PLACE, BOSTON, MA, 02110, US

(73) Assignee: Wyeth, Madison, NJ (US)

(21) Appl. No.: 12/234,033

(22) Filed: Sep. 19, 2008

Related U.S. Application Data
(60) Provisional application No. 60/974,372, filed on Sep. 21, 2007.

(51) Int. Cl. C07D 487/22

(52) U.S. Cl. 540/579

(57) ABSTRACT

The present invention relates to improved methods of resolution and recrystallization for synthesizing compounds useful as 5HT_{2C} agonists or partial agonists, including intermediates thereto.

### CROSS-REFERENCE TO RELATED APPLICATIONS

The present invention claims priority to U.S. provisional application Ser. No. 60/974,372, filed Sep. 21, 2007, the entirety of which is hereby incorporated herein by reference.

### FIELD OF THE INVENTION

The present invention relates to methods for synthesizing compounds useful as 5HT_{2C} agonists or partial agonists, derivatives thereof, and to intermediates thereto.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, 156: 1686-1696, 1999; Masand, P. S., Exp. Opin. Pharmacother. I: 377-389, 2000; Whitaker, R., Spectrum Life Sciences. Decision Resources. 2:1-9, 2000).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology 124: 57-73, 1996; Cowen, P. J., et. al., Human Psychopharmacology 10: 385-391, 1995; Rosenzweig-Lipson, S., et. al., ASPET abstract, 2000).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology 37: 265-272, 1998; Fox, S. H., et. al., Experimental Neurology 151: 35-49, 1998). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology 37: 953-955, 1998; Di Matteo, V., et. al., Neuropharmacology 38: 1195-1205, 1999; Di Giovanni, G., et. al., Synapse 35: 53-61, 2000), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

### SUMMARY OF THE INVENTION

As described herein, the present invention provides methods for preparing compounds having activity as 5HT_{2C} agonists or partial agonists. These compounds are useful for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, intellectual deficit associated with Alzheimer's disease, bipolar disorders, depressive disorders, mood episodes, anxiety disorders, adjustment disorders, eating disorders, epilepsy, sleep disorders, migraines, sexual dysfunction, gastrointestinal disorders, obesity, or a central nervous system deficiency associated with trauma, stroke, or spinal cord injury. Such compounds include those of formula I:

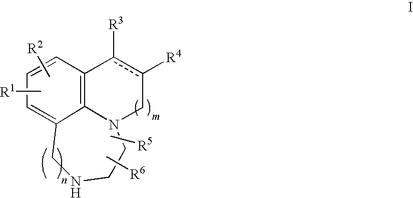

or a pharmaceutically acceptable salt thereof, wherein:
[symbol] designates a single or double bond;
n is 0, 1, or 2;
R¹ and R² are each independently halogen, —CN, phenyl, —R, —OR, —C₁₋₆ perfluoroalkyl, or —OC₁₋₆ perfluoroalkyl;
each R is independently hydrogen or a C₁₋₆ alkyl group;
R³ and R⁴ are taken together to form a saturated or unsaturated 4-8 membered ring, wherein said ring is optionally substituted with 1-3 groups independently selected from halogen, —R, or OR; and
R⁵ and R⁶ are each independently —R.

The present invention also provides synthetic intermediates useful for preparing such compounds. The invention further provides methods of chiral resolution and recrystallization to provide cost effective yields and purity.

### BRIEF DESCRIPTION OF THE DRAWINGS

FIG. 1 shows the X-ray diffraction pattern of Compound A.

FIG. 2 shows the DSC pattern of Compound A.

### DETAILED DESCRIPTION OF CERTAIN EMBODIMENTS

In certain embodiments, the present invention provides a method for preparing compound I-1, (9aR, 12aS)-4,5,6,7,9,9a, 10,11,12,12a-decahydrocyclopenta[c][1,4]diazepino-[6,7,1-ij]quinoline hydrochloride, also known as vabicaserin hydrochloride.

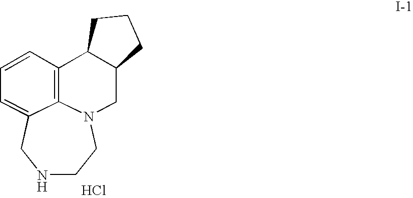

Compound I-1, a potent 5-HT_{2C} agonist, is in detail in U.S. patent application Ser. No 10/422,524, filed Apr. 24, 2003, and International Application WO 03/091250, each of which is incorporated by reference herein in its entirety. Compound I-1 is effective in treating schizophrenia, including the mood disorders or the cognitive impairments associated with schizophrenia.

Certain methods of preparing compounds of the present invention are known in the art and include those described in detail in PCT publication number WO2007/016029 and WO2006/052768, the entirety of each of which is hereby incorporated herein by reference.

In certain embodiments, the present compounds are generally prepared according to Scheme I set forth below:

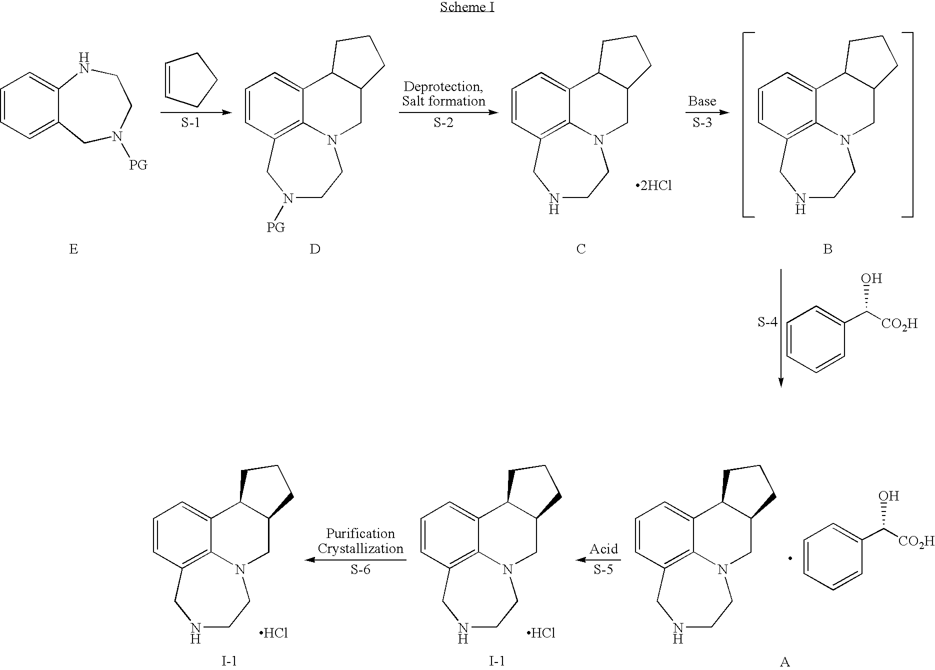

In one aspect, the present invention provides methods for preparing a diastereomerically enriched diastereomeric salt, A, according to the steps depicted in Scheme 1, above. At step S-1, a benzodiazepine of formula E is reacted with formaldehyde, or an equivalent thereof, and pentene in the presence of a Lewis acid. In certain embodiments, the Diels-Alder reaction of benzodiazepine E and pentene in the presence of boron trifluoride etherate provides the cyclopentenyltetrahydroquinoline D.

The PG group of formulae E and D is a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the —NH— moiety to which it is attached, include, but are not limited to, aralkylamines, carbamates, allyl amines, amides, and the like. Examples of PG groups of formulae E and D include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In other embodiments, the PG group of formulae E and D is acetyl.

At step S-2, the amino group is deprotected by removal of PG and a salt complex is formed. One of ordinary skill in the art would recognize that, depending on the choice of PG, deprotection and salt formation may be performed in the same step. For example, when the PG group of formula D is acetyl, contact with certain mineral acids would simultaneously deprotect of the amine group and form an amine salt. Accordingly, in certain embodiments, the present invention provides a method of forming compound C comprising the step of simultaneously deprotecting the amino group and forming an amine salt. Thus, in certain embodiments, the PG group of formula D is an amino protecting group that is removed by alcohols in the presence of strong mineral acids. In certain embodiments, deprotection of an acetyl group and amine salt formation is achieved in the same reaction with ethanol and concentrated hydrochloric acid. In an alternate method, the removal of PG and salt formation at step S-2 may be performed in a stepwise fashion using methods known to one of ordinary skill in the art.

At step S-3, compound C is treated with a suitable base to form the free base compound B. Free bases according to the invention are also prepared, for example, by contacting compound C with a suitable base in the presence of a solvent suitable for free base formation. Such suitable bases include strong inorganic bases, i.e., those that completely dissociate in water under formation of hydroxide anion. In certain embodiments, the base is added in an amount of at least about 1 mol. eq. and, in other embodiments, in an amount of at least about 1 mol. eq. to about 10 mol. eq. relative to compound C. Examples of such bases include alkaline metals, alkaline earth metal hydroxides, and combinations thereof. In other embodiments, the suitable base is sodium hydroxide.

Examples of solvents suitable for use during free base formation at step S-3 include polar solvents such as alkyl alcohols, such as C₁ to C₄ alcohols (e.g. ethanol, methanol, 2-propanol), water, dioxane, or THF (tetrahydrofuran) or combinations thereof. In certain embodiments, the suitable solvent is a C₁ to C₄ alcohol such as methanol, ethanol, 2-propanol, water, or combination thereof According to one aspect of the present invention, aqueous sodium hydroxide is used at step S-3. According to another aspect of the present invention, the free base formation at step S-3 is performed in a bi-phasic mixture of solvents whereby the compound of formula B, as it is formed, is extracted into an organic layer. Thus, a suitable bi-phasic mixture of solvents includes an aqueous solvent and a non-miscible organic solvent. Such non-miscible organic solvents are well known to one of ordinary skill in the art and include halogenated hydrocarbon solvents (e.g. methylene chloride and chloroform), benzene and derivatives thereof (e.g. toluene), esters (e.g. ethyl acetate and isopropyl acetate), and ethers (e.g. t-butylmethyl ether (MTBE), THF and derivatives thereof, glyme, and diglyme) and the like. In certain embodiments, the free base formation at step S-3 is performed in a bi-phasic mixture comprising water, toluene and a suitable aqueous base such as NaOH. In other embodiments, the reaction is performed in a mixture of t-butylmethyl ether and a suitable aqueous base, such as aqueous sodium hydroxide.

At step S-4, the racemic compound B is treated with a chiral agent, mandelic acid, to form a diastereomeric mixture thereof. In certain embodiments, the racemic compound B is treated with a chiral acid, mandelic acid, to form a diastereomeric salt thereof. The resulting diastereomeric mixture is then separated by suitable means to obtain compound A. Such suitable means for separating diastereomeric mixtures are well known to one of ordinary skill in the art and include, but are not limited to, those methods described herein. It will be appreciated that the mandelic acid for use in step S-4 is relatively enantiomerically enriched, i.e., at least about eight-five percent of a single enantiomer of the acid is present. In some embodiments the enantomerically enriched mandelic acid used is S-(+)-mandelic acid.

In certain embodiments, the resulting salt may have about a one-to-one molar mixture of chiral acid to compound B. In certain embodiments the chiral acid is employed in a range from 0.50 to 0.60 mole equivalents relative to compound B. In certain embodiments the chiral acid is employed in a range from 0.50 to 0.55 mole equivalents relative to compound B.

In certain embodiments, each of the aforementioned synthetic steps may be performed sequentially with isolation of each intermediate D, C, B, and A performed after each step. Alternatively, each of steps S-1, S-2, S-3, and S-4, as depicted in Scheme I above, may be performed in a manner whereby no isolation of one or more intermediates D, C, and B is performed.

At step S-5, compound A is transformed from a diastereomeric salt to an enantiomeric salt. One of ordinary skill in the art would recognize that a carboxylate acid moiety in an amine salt similar to compound A may be exchanged with an acid having a pKa lower than the chiral resolving acid to form a desired resolved enantiomeric salt. In certain embodiments, the present invention provides a method of forming compound I-1 by contacting the diastereomeric salt A with a strong mineral acid, i.e. an acid having a pKa less than 1. Examples of such acids include hydrochloric acid, hydrobromic acid, hydroiodic acid, sulfuric acid, phosphoric acid, and combinations thereof. In some embodiments, the acid is an organic acid. Such acids include malic acid, succinic acid, trifluoro acetic acid, acetic acid, methane sulfonic acids, alkyl- and aryl-sulfonic acids and combinations thereof.

In certain embodiments, the acid results in formation of a pharmaceutically acceptable salt. In some embodiments, the acid is hydrochloric acid. Suitable solvents for forming the enantiomeric salt include polar solvents such as ethanol, methanol, isopropyl acetate, ethyl acetate, isopropanol, n-propanol, n-butanol, tetrahydrofuran, acetonitrile, and combinations thereof. In certain embodiments, compound A is treated with hydrochloric acid in ethyl acetate to form the enantiomeric dihydrochloride salt thereof. In certain embodiments, compound A is treated with hydrochloric acid in ethyl acetate to form the enantiomeric monohydrochloride salt thereof.

At step S-6, compound I-1 is recrystallized to further enrich the chemical purity and optical purity, or enantiomeric excess (ee), of compound I-1. The present inventors have surprisingly discovered that recrystallization from a ternary solvent mixture results in increased enantiomeric excess. In addition, it was surprisingly found that use of the ternary solvent mixture, in accordance with the present invention, results in higher yields of compound I-1 compared with other solvent mixtures.

In certain embodiments, an anti-solvent is employed during crystallization. As used herein, the term “anti-solvent” refers to a solvent in which the crystalline compound has limited or poor solubility. In certain embodiments the anti-solvent is selected from ethyl acetate, acetone, methyl ethyl ketone, toluene, isopropyl acetate, and t-butyl methyl ether. In some embodiments, the anti-solvent is t-butyl methyl ether.

Those skilled in the art will appreciate the unpredictable nature of recrystallization, in that one cannot predict, calculate, or assume a priori that any particular combination of solvents or anti-solvents will engender or afford a crystalline product. The variables and techniques that may be employed to develop and optimize a crystallization process are numerous, including, but not limited to solvent choice, temperature, addition of anti-solvents, rate of addition of anti-solvents, agitation, and seeding. Crystal structure (polymorphism) and crystal shape (morphology) may also be affected by subtle differences in crystallization conditions. The inventive method described herein resulted in part from the discovery that certain ternary solvent mixtures afford the step S-6 recrystallization of compound I-1 in substantially higher yields and higher enantiomeric excess (ee) than other ternary or binary solvent mixtures. In some embodiments, compound I-1 has a % ee of at least 99.5%. In other embodiments, compound I-1 has a % ee of at least 99.85%.

In certain embodiments, the yield of step S-6 is at least about 50%. In certain embodiments, the yield of step S-6 is at least about 60%. In certain embodiments, the yield of step S-6 is at least about 70%. In certain embodiments, the yield of step S-6 is at least about 77%. In certain embodiments, the yield of step S-6 is at least about 85%.

In certain embodiments, the % ee of compound of formula I-1 following step S-6 is at least about 85%. In certain embodiments, the % ee of compound of formula I-1 following step S-6 is at least about 90%. In certain embodiments, the % ee of compound of formula I-1 following step S-6 is at least about 95%. In certain embodiments, the % ee of compound of formula I-1 following step S-6 is at least about 99%. In certain embodiments, the % ee of compound of formula I-1 following step S-6 is at least about 99.99%.

As used herein, the term “diastereomeric salt” refers to the adduct of a chiral compound with a chiral acid. As used herein, the term “diastereomerically enriched,” as used herein signifies that one diastereomer makes up at least 80% or 85% of the preparation. In certain embodiments, the term diastereomerically enriched signifies that at least 90% of the preparation is one of the diastereomers. In other embodiments, the term signifies that at least 95% of the preparation is one of the diastereomers. In yet other embodiments, the term signifies that at least 99.5% of the preparation is one of the diastereomers.

As used herein, the term “enantiomeric salt” refers to the salt of the resolved chiral compound wherein the compound is enriched in one enantiomer. As used herein, the term “enantiomerically enriched,” as used herein signifies that one enantiomer makes up at least 80% or 85% of the preparation. In certain embodiments, the term enantiomerically enriched signifies that at least 90% of the preparation is one of the enantiomers. In other embodiments, the term signifies that at least 95% of the preparation is one of the enantiomers. In yet other embodiments, the term signifies that at least 99.5% of the preparation is one of the enantiomers.

In certain embodiments, the present invention provides a method comprising the steps of:
(a) providing compound I-1 having an initial purity and % enantiomeric excess:

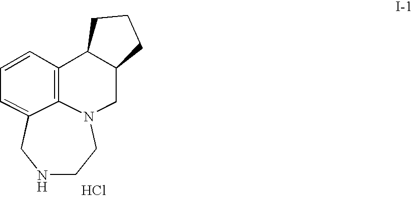

and
(b) recrystallizing compound I-1 from a ternary solvent system to provide compound I-1 with increased purity and % enantiomeric excess.

The resulting enantiomeric salt I-1 may be isolated by techniques known to those skilled in the art such as by crystallization followed by separation of the crystals. For example, in one embodiment the resulting mixture of enantiomeric salt may be cooled gradually to form crystals of the enantiomeric salt, followed by filtration to isolate the crystals. The isolated crystals may then optionally be recrystallized to increase purity. For example, in some embodiments, the isolated crude enantiomeric salt is mixed in a suitable solvent and heated to dissolve the enantiomeric salt. The mixture is then gradually cooled to effect crystallization. Examples of suitable solvents from which the enantiomeric salts are recrystallized include protic solvents such as C₁-C₄ alcohols including ethanol, methanol, isopropanol, n-propanol, n-butanol; water miscible polar aprotic solvents such as tetrahydrofuran, dioxan, acetone, acetonitrile; water; and combinations thereof.

In certain embodiments, the recrystallization solvent used is a C₁ to C₄ alcohol or mixtures of C₁ to C₄ alcohols with water. In some embodiments, the recrystallization solvent is ethanol, in about 5 parts by volume based on volume of the compound. In other embodiments, the ethanol is mixed with 0-15% water based on volume of ethanol. In certain embodiments, the recrystallization solvent is ethanol mixed with about 8% water by volume of ethanol. Without wishing to be bound by any particular theory, it is believed that the presence of water increases the throughput (i.e., yield) by solubilizing the compound.

In accordance with the present invention, an anti-solvent is employed during crystallization. In certain embodiments the anti-solvent is selected from ethyl acetate, acetone, methyl ethyl ketone, toluene, benzene, isopropyl acetate, and t-butyl methyl ether. In one embodiment, the anti-solvent is t-butyl methyl ether. In some embodiments, the t-butyl methyl ether is added in about 2 parts based on volume of the compound. In some embodiments, the t-butyl methyl ether is added in about 10 parts based on volume of the compound.

In some embodiments, crystallization of compound I-1 in a ternary solvent system, in accordance with the present invention, results in an increase in % enantiomeric excess from about 92% to about 99.8%. In certain embodiments, such crystallization method results in a chemical purity of about 99.4% and a chiral purity of at least about 99.99%.

One of ordinary skill in the art will appreciate that crystallizations may employ a seeding step. In certain embodiments, the crystallization step further includes the step of seeding. In other embodiments, the crystallization step is performed in the absence of a seeding step.

In certain embodiments, processes of the present invention further including a co-milling step. In some embodiments, the co-milling step results in compound I-1 having a particle size range wherein about 10% of the particles are about 3.57 micron, about 50% of the particles are about 19.41 microns, and about 90% of the particles are about 65.31 microns.

According to another aspect, the present invention provides a method for preparing compound I-1:

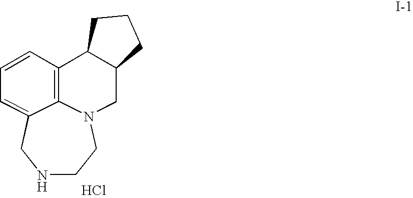

comprising the steps of:
(a) providing compound A:

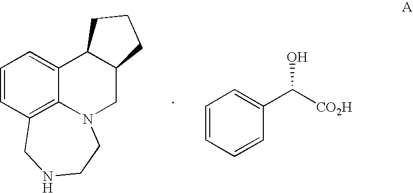

and
(b) treating said compound A with hydrochloric acid to form compound I-1.

In certain embodiments, compound A is treated with hydrochloric acid in ethyl acetate to form compound I-1.

According to another embodiment, the present invention provides a method for preparing diasteromerically enriched compound A:

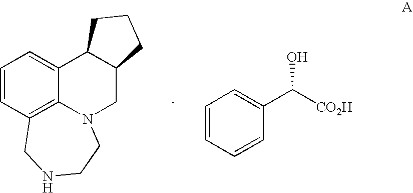

comprising the steps of:
(a) providing compound B:

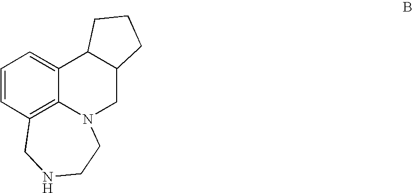

and
(b) treating said compound B with S-(+)-mandelic acid to form compound A-1:

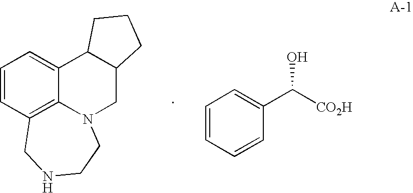

and
(c) obtaining said compound A by suitable physical means.

As described herein, the chiral acid is enantiomerically enriched mandelic acid. In certain embodiments, the chiral acid is S-(+)-mandelic acid.

In certain embodiments, the chiral acid is R-(−)-mandelic acid. Thus, another aspect of the present invention provides a compound of formula A-2:

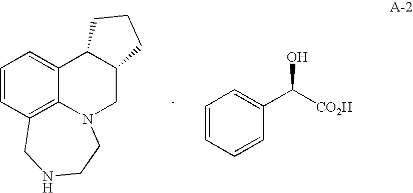

comprising the steps of:
(b) providing compound B:

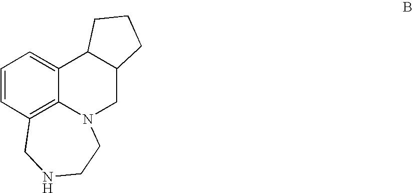

and
(b) treating said compound B with R-(−)-mandelic acid to form compound A-3:

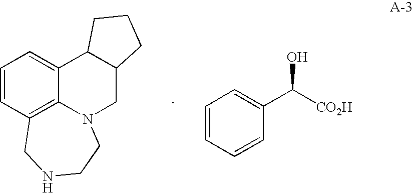

and
(c) obtaining said compound A-2 by suitable physical means.

The term “separated by suitable physical means” refers to methods of separating enantiomeric or diastereomeric mixtures. Such methods are well known in the art and include preferential crystallization, distillation, and trituration, among others. Chiral agents and separation methods are described in detail in Stereochemistry of Organic Compounds, Eliel, E. L. and Wilen, S. H., 1994, published by John Wiley and Sons.

In certain embodiments, a diastereomeric salt A is obtained via preferential crystallization of a diastereomeric salt formed at step (b) above. In other embodiments, the crystallization is achieved from a protic solvent. In still other embodiments, the protic solvent is an alcohol. It will be appreciated that the crystallization may be achieved using a single protic solvent or a combination of one or more protic solvents. Such solvents and solvent mixtures are well known to one of ordinary skill in the art and include one or more straight or branched alkyl alcohols. In certain embodiments, the crystallization is achieved from ethanol. In certain embodiments, the crystallization is achieved from a mixture of ethanol and ethyl acetate. Without wishing to be bound by any particular theory, it is believed that the presence of ethyl acetate increases the throughput (i.e., yield) by solubilizing the compound.

In some embodiments, compound A has a % enantiomeric excess of about 92%. In some embodiments, compound A has a % enantiomeric excess of about 98%.

In certain embodiments, compound A comprises an equimolar amount of chiral acid and amine. In other embodiments, compound A comprises a substoichiometric amount of chiral acid. As used herein, the term “substoichiometric amount” denotes that the chiral acid is used in less than 1 mole equivalent relative to the compound B. In certain embodiments the chiral acid is employed in a range from 0.50 to 0.60 mole equivalents relative to compound B. In certain embodiments the chiral acid is employed in a range from 0.50 to 0.55 mole equivalents relative to compound B.

It should be readily apparent to those skilled in the art that diastereomeric enrichment of one diastereomer in the crystallized compound A causes a diastereomeric enrichment in the mother liquor of the other diastereomeric form. Therefore, according to another embodiment, the invention relates to a method of enhancing the % de of diastereomerically enriched compound A as compared with compound A-1. As used herein, the term “% de” refers to the percent diastereomeric excess as would be understood by one of ordinary skill in the art. Similarly, as used herein, the term “% ee” refers to the percent enantiomeric excess as would be understood by one of ordinary skill in the art.

It is contemplated that Compound A can be provided in a variety of physical forms. For example, Compound A can be put into solution, suspension, or be provided in solid form. When Compound A is in solid form, said compound may be amorphous, crystalline, or a mixture thereof.

In some embodiments, the present invention provides crystalline Compound A. In some embodiments, Compound A is characterized in that it has one or more, two or more, or three or more, peaks in its XRPD pattern selected from those at about 6.0, 6.6, 8.1, 11.6, 13.2, 15.2, 16.1, 20.5 and 24.9 degrees 2-theta. As used herein, the term “about”, when used in reference to any degree 2-theta value recited herein, refers to the stated value±0.2 degree 2-theta.

In other embodiments, crystalline Compound A is characterized in that is has substantially all of the peaks in its XRPD pattern listed in Table 1, below.

TABLE 1
PEAK: 21(pts)/Parabolic Filter, Threshold = 0.0, Cutoff =
5.0%, BG = 3/1.0, Peak − Top = Summit

| 2-Theta | d(Å) | BG | Height | H % | Area | A % | FWHM |
|---|---|---|---|---|---|---|---|
| 5.978 | 14.7727 | 329 | 2762 | 30.2 | 61703 | 60.2 | 0.38 |
| 6.601 | 13.3802 | 372 | 9149 | 100 | 102454 | 100 | 0.19 |
| 8.08 | 10.9328 | 222 | 1439 | 15.7 | 26065 | 25.4 | 0.308 |
| 11.619 | 7.6097 | 216 | 1800 | 19.7 | 26121 | 25.5 | 0.247 |
| 13.219 | 6.6922 | 216 | 2886 | 31.5 | 35619 | 34.8 | 0.21 |
| 14.94 | 5.925 | 257 | 567 | 6.2 | 15981 | 15.6 | 0.479 |
| 15.239 | 5.8093 | 257 | 985 | 10.8 | 29445 | 28.7 | 0.508 |
| 15.5 | 5.7121 | 257 | 750 | 8.2 | 14093 | 13.8 | 0.319 |
| 16.1 | 5.5005 | 257 | 2539 | 27.8 | 29641 | 28.9 | 0.198 |
| 16.603 | 5.3352 | 257 | 414 | 4.5 | 9729 | 9.5 | 0.399 |
| 17.14 | 5.1691 | 257 | 566 | 6.2 | 8409 | 8.2 | 0.253 |
| 17.937 | 4.9411 | 392 | 698 | 7.6 | 7987 | 7.8 | 0.195 |
| 18.757 | 4.727 | 401 | 861 | 9.4 | 14604 | 14.3 | 0.288 |
| 19.377 | 4.5772 | 423 | 767 | 8.4 | 10526 | 10.3 | 0.233 |
| 19.9 | 4.458 | 482 | 797 | 8.7 | 7096 | 6.9 | 0.151 |
| 20.481 | 4.3329 | 470 | 2325 | 25.4 | 30161 | 29.4 | 0.221 |
| 20.839 | 4.2592 | 462 | 454 | 5 | 6488 | 6.3 | 0.243 |
| 21.139 | 4.1995 | 434 | 604 | 6.6 | 6569 | 6.4 | 0.185 |
| 21.429 | 4.1433 | 470 | 162 | 1.8 | 790 | 0.8 | 0.078 |
| 22.158 | 4.0086 | 428 | 226 | 2.5 | 3250 | 3.2 | 0.23 |
| 22.858 | 3.8873 | 469 | 891 | 9.7 | 16375 | 16 | 0.313 |
| 23.357 | 3.8054 | 478 | 383 | 4.2 | 4274 | 4.2 | 0.178 |
| 24 | 3.7049 | 529 | 738 | 8.1 | 11603 | 11.3 | 0.267 |
| 24.899 | 3.5731 | 278 | 1234 | 13.5 | 23687 | 23.1 | 0.326 |
| 26.56 | 3.3534 | 276 | 471 | 5.1 | 6577 | 6.4 | 0.238 |
| 27.42 | 3.2501 | 303 | 535 | 5.9 | 6557 | 6.4 | 0.208 |
| 29.303 | 3.0453 | 293 | 345 | 3.8 | 5843 | 5.7 | 0.288 |

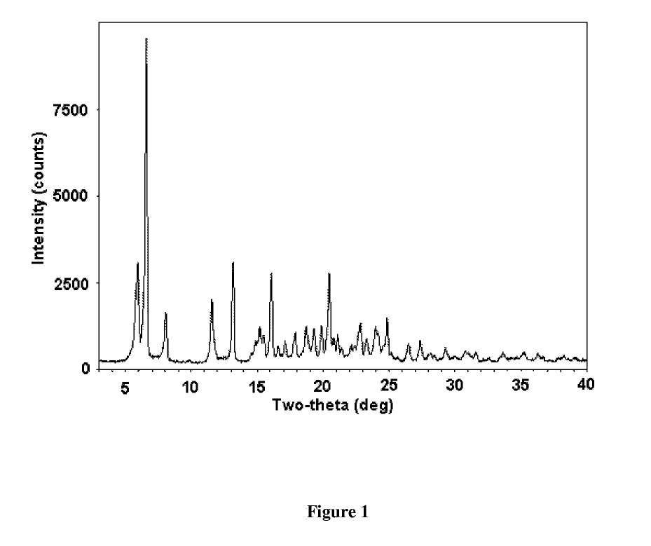

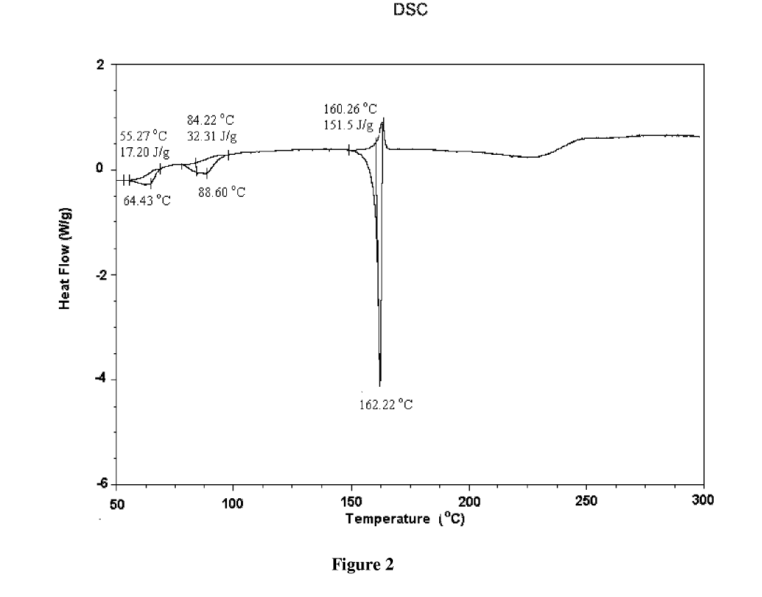

In some embodiments, the present invention provides crystalline Compound A, have an X-ray diffraction pattern substantially similar to that depicted in FIG. 1. In some embodiments, the present invention provides crystalline Compound A, have a DSC pattern substantially similar to that depicted in FIG. 2. In some embodiments, crystalline Compound A has a melting point of about 162° C.

According to another embodiment, the present invention provides a method of obtaining compound B:

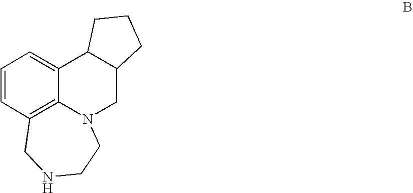

comprising the steps of:
(a) combining compound C:

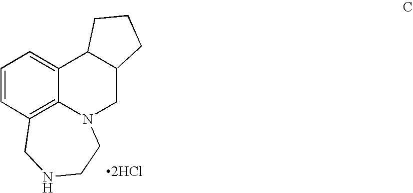

with a suitable solvent; and
(b) treating said compound C with a base to give free base compound B.

The dihydrochloride salt C can be contacted with base to form the corresponding free base compound B. Preferably the dihydrochloride salt and base are combined in the presence of a suitable solvent in which the dihydrochloride salt is at least partially soluble in such as hot (about 60 to 80° C.) water, polar solvents such as alkyl alcohols, such as C₁ to C₄ alcohols (e.g. ethanol, methanol, 2-propanol), dioxane, or THF (tetrahydrofuran) or combinations thereof to form the corresponding free base. The base is preferably added in an amount of at least about 2 mol. eq. and more preferably in an amount of at least about 2 mol. eq. to about 3 mol. eq. relative to the dihydrochloride salt C. Suitable bases include alkaline metal hydroxides or alkaline earth metal hydroxides, carbonates or phosphates, as well as organic bases and combinations thereof. In certain embodiments, the base is sodium hydroxide. The free base once formed may optionally be extracted using an extraction solvent. One of ordinary skill in the art will understand that extraction solvents include solvents which are immiscible with water and have at least partial solubility with compound B. In some embodiments, the extraction solvent is t-butylmethyl ether.

According to another aspect of the present invention, the free base formation is performed in a bi-phasic mixture of solvents whereby compound B, as it is formed, is extracted into an organic layer. Thus, a suitable bi-phasic mixture of solvents includes an aqueous solvent and a non-miscible organic solvent. Such non-miscible organic solvents are well known to one of ordinary skill in the art and include halogenated hydrocarbon solvents (e.g. methylene chloride and chloroform), benzene and derivatives thereof (e.g. toluene), esters (e.g. ethyl acetate and isopropyl acetate), and ethers (e.g. t-butylmethyl ether (MTBE), THF and derivatives thereof, glyme, and diglyme) and the like. In certain embodiments, the free base formation at step (b) is performed in a bi-phasic mixture comprising water and toluene. In other embodiments, the suitable base is water soluble such that the reaction is performed in a mixture of t-butylmethyl ether and a suitable aqueous base, such as aqueous sodium hydroxide.

In other embodiments, the present invention provides a method comprising the steps of:
(a) providing a compound of formula D:

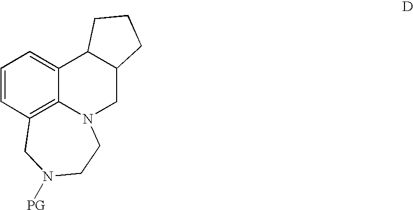

wherein, PG is a suitable amino protecting group,
and
(b) treating said compound of formula D with hydrochloric acid to give amine salt C:

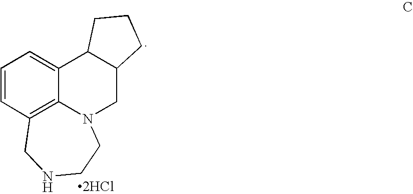

In certain embodiments, the transformation of a compound of formula D to compound C is performed in the presence of a suitable solvent. Suitable solvents include protic solvents such as alkanols and polar aprotic solvents which are miscible with water, such as dioxan or glyme and combinations thereof. Further examples of protic solvents include acetic acid or C₁-C₄ alcohols. Certain embodiments include a mixture of ethanol and ethyl acetate.

The amino group of compound D is deprotected by removal of PG and a salt complex is formed. One of ordinary skill in the art would recognize that, depending on the choice of PG, deprotection and salt formation may be performed in the same step. For example, when the PG group of formula D is acetyl, contact with certain mineral acids would simultaneously deprotect of the amine group and form an amine salt. Accordingly, in certain embodiments, the present invention provides a method of forming compound C comprising the step of simultaneously deprotecting the amino group and forming an amine salt. Thus, in certain embodiments, the PG group of formula D is an amino protecting group that is removed by alcohols in the presence of strong mineral acids. In certain embodiments, deprotection of an acetyl group and amine salt formation is achieved in the same reaction with ethanol and concentrated hydrochloric acid. In an alternate method, the removal of PG and salt formation may be performed in a stepwise fashion using methods known to one of ordinary skill in the art.

In certain embodiments, the present invention provides a compound of formula D:

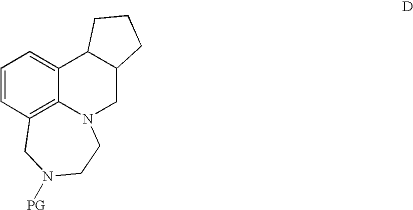

comprising the steps of:
(a) combining a compound of formula E:

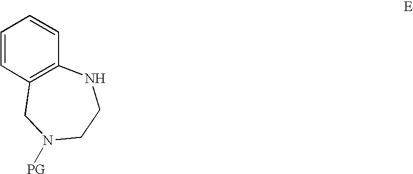

wherein, PG is a protecting group,
with a suitable solvent to form a mixture thereof; and
(b) treating said compound of formula E with formaldehyde and pentene in the presence of a Lewis acid to give compound of formula D.

The PG group of formulae E and D is a suitable amino protecting group. Suitable amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups, taken with the —NH— moiety to which it is attached, include, but are not limited to, aralkylamines, carbamates, allyl amines, amides, and the like. Examples of PG groups of formulae E and D include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, and the like. In certain embodiments, the PG group of formulae E and D is acetyl.

In certain embodiments, the Lewis acid is boron trifluoride etherate.

In certain embodiments, the solvent is acetonitrile.

In certain embodiments, the Diels-Alder reaction of benzodiazepine E and pentene in the presence of boron trifluoride etherate provides the cyclopentenyltetrahydroquinoline D, wherein PG is acetyl.

According to one embodiment, step (b) above is performed using aqueous formaldehyde. According to another embodiment, step (b) is performed using a formaldehyde equivalent. Such formaldehyde equivalents are well known to one of ordinary skill in the art. In some embodiments, the formaldehyde equivalent is added in solid form to the reaction solvent to form a reaction suspension or the solid formaldehyde equivalent may be suspended in a reaction solvent and added to the reaction mixture. In other embodiments, paraformaldehyde is used as the formaldehyde equivalent, and is added in amounts sufficient to consume the compound of formula E. In certain embodiments, paraformaldehyde is in a solid form such as powder or prills. In certain embodiments, the use of paraformaldehyde prills yields less of the dimer by products F-2 and F-3 (infra) than other formaldehyde equivalents. In some embodiments, paraformaldehyde is added in amounts of at least about 0.90 mole equivalents, in amounts of about 0.90 mole equivalents to about 1.10 mole equivalents, or in amounts of from about 1.0 mole equivalents to about 1.05 mole equivalents relative to the compound of formula E.

In certain embodiments, steps S-1 and S-2 are performed without isolation of Compound D. It was surprisingly found that such process is advantageous. Specifically, it was found that performing steps S-1 and S-2 without isolating Compound D results in an improved yield of Compound C of 75% compared to 58% for the two-step process where Compound D was isolated. Performing steps S-1 and S-2 without isolation of Compound D also results in an improved throughput of 11% over the 6.4% throughput of the two-step process, and drastically reduces cycle time by 3-5 weeks over the two-step process.

According to another embodiment, the present invention provides compound I-1 substantially free of compounds F-1, F-2, and F-3:

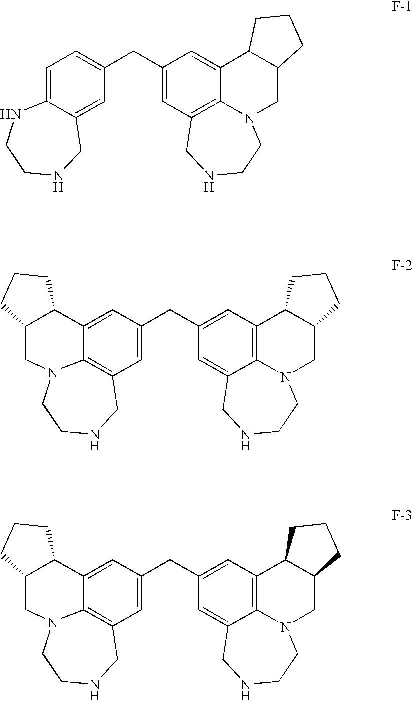

Compounds F-1, F-2, and F-3 were identified as impurities arising from the step S-1 Diels-Alder reaction. “Substantially free,” as used herein, means that at least about 80% by weight of the desired compound is present. In other embodiments, at least about 92% by weight of a desired compound is present. In still other embodiments of the invention, at least about 99% by weight of a desired compound is present. Such impurities may be isolated from product mixtures by any method known to those skilled in the art, including high performance liquid chromatography (HPLC).

In certain embodiments, the present invention provides a composition comprising compound I-1 and one or more of compounds F-1, F-2, and F-3.

In certain embodiments, the compounds A and I-1 described in Scheme 1 are provided substantially free of the corresponding enantiomer. “Substantially free,” as used herein, means that the compound is made up of a significantly greater proportion of one enantiomer. In other embodiments, at least about 95% by weight of a desired enantiomer is present. In still other embodiments of the invention, at least about 99%, at least about 99.5%, or at least about 99.85% by weight of a desired enantiomer is present. Such enantiomers may be isolated from racemic mixtures by any method known to those skilled in the art, including high performance liquid chromatography (HPLC) and chiral salt resolution, or prepared by methods described herein.

In some embodiments, the present invention provides compound I-1 having total impurities of less than 0.5%, less than 0.4%, or less than 0.3% by weight. In some embodiments, the present invention provides compound I-1 having less than 0.2% of any one of compounds F-1, F-2, and F-3. In certain embodiments, the present invention provides compound I-1 having less than 0.15% of any one of compounds F-1, F-2, and F-3.

The present invention provides methods that provide enantiomerically enriched compound I-1 in substantially higher yields than described previously (U.S. patent application Ser. No. 10/422,524, filed Apr. 24, 2003, and International Application WO 03/091250).

### EXAMPLES

As indicated herein, the % enantiomeric excess data was obtained via the following chiral HPLC method:
Column: Chirobiotic V column (Astec) 4.6 mm×150 mm
Mobile Phase: 0.9 g ammonium trifluoroacetate in 1 L of methanol
Flow rate: 0.3 mL/minute
Temperature: 10° C.
Time: 12 minutes
Wavelength: 215 nm

As indicated herein, the % purity data was obtained via the following chiral HPLC method:
Column: Chromolith Performance RP-18e (100×4.6 mm)
Mobile Phase: A=95:5:0.1 water:CH₃CN:H₃PO₄
B=95:5:0.1 CH₃CN:water:H₃PO₄
Gradient: 5% B to 95% B over 8 minutes
Flow rate: 1 mL/minute
Temperature: Ambient
Time: 10 minutes
Wavelength: 210 nm

### Example 1

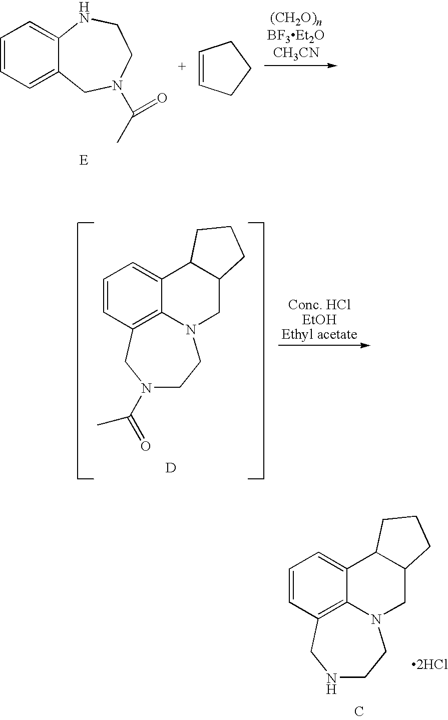

To a mixture of compound E (160.0 g, 0.84 mol), paraformaldehyde prills (25.2 g, 0.84 mol), cyclopentene (342.0 g, 5.05 mol) in acetonitrile (696.0 g, 880 mL) at 15° C. was added borontrifluoride diethyl etherate solution (328.0 g, 288 mL, 2.27 mol) via addition funnel. The reaction mixture was heated at 35° C. for 8 h. The reaction mixture was cooled to 15° C. and aqueous sodium hydroxide solution was added (a mixture of 546.0 g of 50% aq. sodium hydroxide and water 546.0 g). The mixture was stirred at 25° C. for 3 h. The top organic layer was filtered, separated, washed with brine (200 mL), and concentrated to a volume of 350 mL. Ethyl acetate (1.08 kg) was added, the layers were separated and the organic layer was washed with water (320 mL). The organics were concentrated to a volume of 350 mL, ethanol (1.00 L) was added and concentrated again to 450 mL. Concentrated HCl (194.0 g) was added and the resulting suspension was heated at reflux (82° C.) for 12 h and cooled to 65° C. Ethyl acetate (0.650 kg) was added and the mixture cooled to 20° C. and stirred for 6 h. The resulting solids were filtered and washed with ethyl acetate (0.270 kg). The solid product was dried in a vacuum oven with a nitrogen bleed at 45° C. for a minimum of 6 h to give a dry weight of 190.0 g (75%) of compound C.

### Example 2

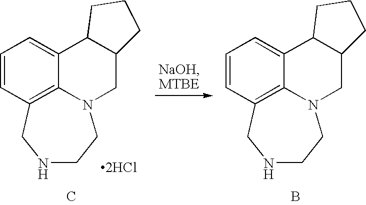

A mixture of compound C (0.20 kg, 0.600 mol) in water (0.60 L) was stirred and heated to 50° C., producing a hazy brown solution. To this, a solution of sodium hydroxide (0.110 L of 50% aq. NaOH in 0.062 L additional water) was added via addition funnel over 5 min, maintaining temperature in the range of 50-60° C. The resulting clear/hazy solution was stirred at 65-75° C. for 15 min to afford a clear solution. The contents were then cooled to 37° C., producing a clear/hazy solution, at which time t-butylmethyl ether (MTBE, 0.300 L) was added via addition funnel over 2 min, maintaining temperature in the range of 30-40° C. The resulting biphasic mixture was stirred for 30 min, cooled to 22° C., and stirred for an additional 10 min, forming 2 clear layers. Layers were then separated, the organic layer washed with sat. NaCl solution (0.10 L) and separated. Ethanol (0.40 L) was added to the organic layer to form a clear solution which was concentrated by atmospheric distillation to a volume of 0.34 L. The resulting clear solution of compound B was used without further manipulation or isolation.

### Example 3

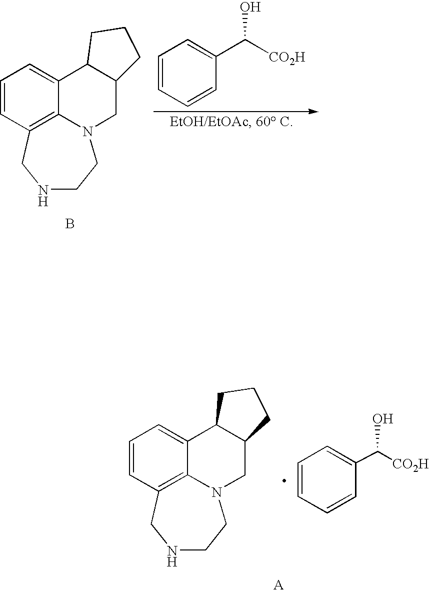

A solution of resolving agent was prepared by mixing S-(+)-mandelic acid (0.054 kg, 0.036 mol) in ethanol (0.220 L). To a crude solution of compound B at 55° C., ethanol (0.30 L) was added via addition funnel. To this, S-(+)-mandelic acid solution (0.260 L) was added via addition funnel over 15 min. to form a suspension. The mixture was heated to 60-70° C. until all solids dissolved and stirred for 15 min. The contents of the reaction were cooled to 57° C. over 30 min, forming a hazy solution, with continued stirring for 60 min. Ethyl acetate (0.46 L) was then added via addition funnel over 30 min, maintaining temperature in the range of 50-60° C. and the suspension stirred for an additional 60 min. The mixture was cooled to 21° C. over 60 min and then stirred for an additional 2 h, forming a thick suspension. The solids were vacuum filtered over polypropylene cloth using a Buchner funnel under house vacuum, and the solids rinsed with ethyl acetate (0.440 L). The solids were dried in a vacuum oven at 50° C. to yield 0.102 kg (41%) of compound A. The XRPD pattern of compound A is depicted in FIG. 1. The DSC pattern of compound A is depicted in FIG. 2.

Analytical table for compound A:

| Test | Found |
|---|---|
| HPLC | |
| Purity A (area %, t_{R} = 6.0 min) | 96.6% |
| Compound F-2 | 0.17% |
| Compound F-3 | 0.12% |
| Mandelic acid | 3.2% |
| Residual Solvents | |
| Ethanol | 3.8% |
| Ethyl Acetate | 0.0072% |

t_{R} = retention time

### Example 4

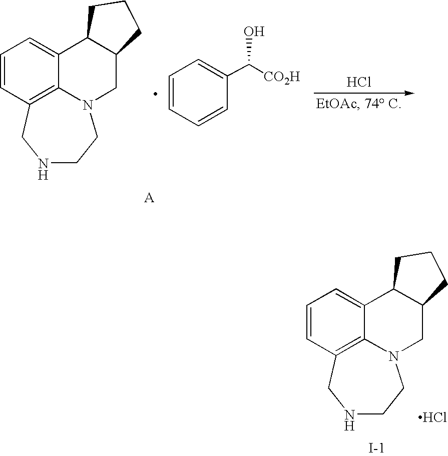

A solution of hydrochloric acid in ethyl acetate was prepared by the addition of concentrated hydrochloric acid (52.0 mL, 0.63 mol) to ethyl acetate (1.840 L). The resulting solution was then added to benzodiazepine A (200 g, 0.525 mol) to form a thick suspension. The resulting mixture was heated with stirring to 74° C. over 40 min. and heating continued for 3 h. The reaction mixture was then cooled to 25° C. and vacuum filtered using a Buchner funnel. The solid residue I-1 was washed with ethyl acetate (500 mL) and dried under house vacuum for 3 h.

### Example 5

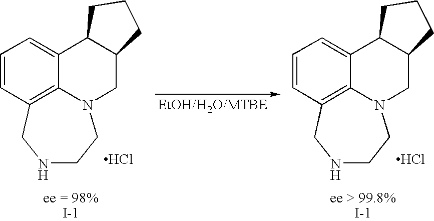

To a suspension of compound I-1 (50.0 g, 0.20 mol) in 250 mL SDA-35 ethanol (i.e., 8% water by volume), 16 mL of water was added, and the resulting mixture was heated with stirring to 75° C. over 30 min, during which time an additional 50 mL ethanol was added. After the formation of a clear solution, the solution was cooled with stirring to 62° C. over 1 h. The contents of the solution were clarified through a filter paper under vacuum while maintaining the solution at 50 to 60° C. throughout. The filtrate was then heated with stirring to 70° C. over 40 min., and then cooled to 50° C. over a minimum of 1.5 h while agitating (rate controlled at 0.33° C./min.) to form a thin suspension. The temperature was held at 46° C. for 1 hour after crystallization established. The mixture was then cooled to 25° C. over 1 h, at which time t-butylmethyl ether (TBME, 600 mL) was added via additional funnel over a minimum of 1.5 h while maintaining the temperature in the range of 23-28° C. The mixture was then cooled to 8° C. over a minimum of 1 h. The mixture was then vacuum filtered using a Buchner funnel, and the solid residue washed with a mixture of ethanol:TMBE (187 mL, 1:3) at 3 to 8° C. The solid product was dried in a vacuum oven with a nitrogen bleed at 50° C. for a minimum of 10 h to give a dry weight of 37.0 g (74%).

### Analytical Table for Compound I-1 (Post-recrystallization)

| Test | Found |
|---|---|
| HPLC | |
| Purity (area %) | 99.83% |
| Enantiomer I-1 (t_{R} = 9.4 min) | 100 |
| Enantiomer I-2 (t_{R} = 9.0 min) | None detected |
| Compound F-1 | 0.055% |
| Compound F-2 | 0.117% |
| Compound F-3 | 0.17% |
| Melting Point | 257° C. |
| Residual Solvents | |
| Ethanol | 0.083% |
| TBME | 0.007% |
| Ethyl Acetate | None detected |

t_{R} = retention time

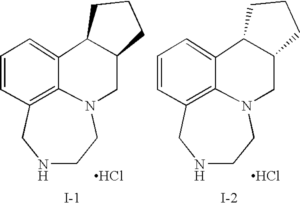

### Example 6

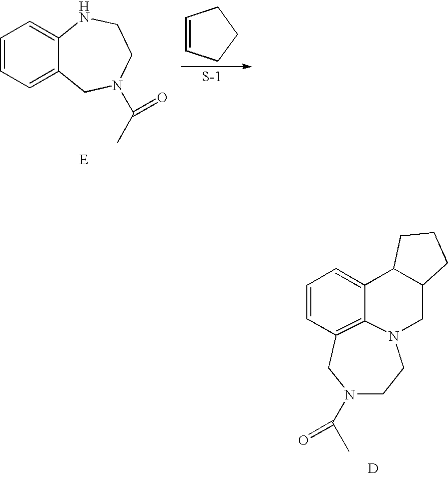

The Diels-Alder reaction, step S-1, was carried out using various conditions. Specifically, the reaction was performed while varying the formaldehyde source and the Lewis Acid. Results of these experiments are shown in the table below, where each reaction was performed in acetonitrile.

Yields for step S-1

| paraformaldehyde | | | temp | % product |
|---|---|---|---|---|
| type | Lewis acid | time | (° C.) | conversion |
| prills | BF₃ etherate | 21-23 h | 15-25 | 97 |
| powder | BF₃ etherate | 21-23 h | 15-25 | 94 |
| trioxane | BF₃ etherate | 21-23 h | 15-25 | 85 |
| prills | AlCl₃ | 17 h | 35 | 61 |
| prills | BiCl₃ | 17 h | 35 | 92 |
| prills | Cu(OTf)₂ | 17 h | 35 | 43 |
| prills | InCl₃ | 17 h | 35 | 27 |
| prills | BCl₃ | 17 h | 35 | 100 |
| prills | BF₃ etherate | 17 h | 35 | 85 |
| prills | TiCl₄ | 17 h | 35 | 100 |
| prills | Sc(OTf)₃ | 17 h | 35 | 66 |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method comprising the steps of:
(a) providing compound I-1 having an initial purity and percent enantiomeric excess: 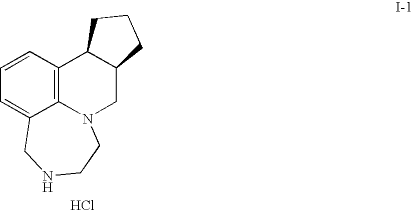
and
(b) recrystallizing compound I-1 from a ternary solvent system to provide compound I-1 with increased purity and % enantiomeric excess.

2. The method according to claim 1, wherein the ternary solvent system comprises tert-butyl methyl ether.

3. The method according to claim 2, wherein the t-butyl methyl ether is added in about 2 parts by volume of the compound I-1.

4. The method according to claim 2, wherein the ternary solvent system comprises ethanol, water, and tert-butyl methyl ether.

5. The method according to claim 1, further comprising the step of:
(a) providing compound A: 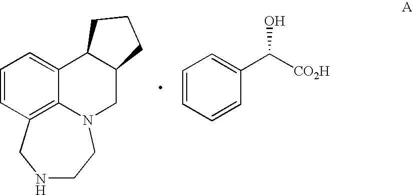
and
(b) treating said compound A with hydrochloric acid to form compound I-1.

6. The method according to claim 5, wherein compound A is treated with hydrochloric acid in ethyl acetate.

7. The method according to claim 5, further comprising the steps of:
(a) providing compound B: 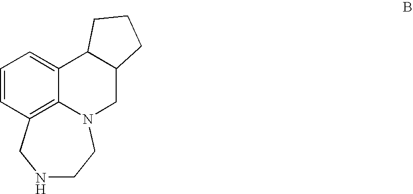
(b) treating said compound B with S-(+)-mandelic acid to form compound A-1: 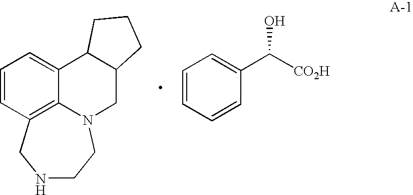
and
(c) obtaining said compound A by suitable physical means.

8. The method according to claim 7, wherein the suitable physical means is preferential crystallization.

9. The method according to claim 8, wherein the S-(+)-mandelic acid is present in a range from 0.50 to 0.60 mole equivalents.

10. The method according to claim 9, wherein the S-(+)-mandelic acid is present in a range from 0.50 to 0.55 mole equivalents.

11. The method according to claim 8, wherein compound A is diastereomerically enriched.

12. The method according to claim 7, further comprising the step of
(a) providing compound C: 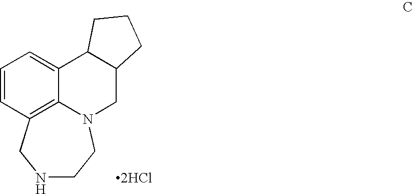
optionally combining compound C with a suitable solvent; and
(b) treating said compound C with a base to give free base compound B.

13. The method according to claim 12, wherein the base is sodium hydroxide.

14. The method according to claim 13, wherein the suitable solvent is a biphasic solvent mixture.

15. The method according to claim 12, further comprising the steps of:
(a) providing a compound of formula D: 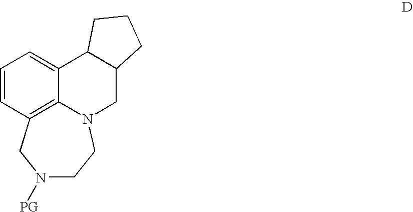
wherein, PG is a suitable amino protecting group,
and
(b) treating said compound of formula D with hydrochloric acid to give amine salt C:

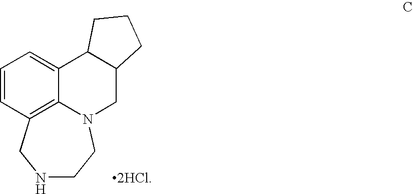

16. The method according to claim 12, further comprising the steps of:
(a) providing a compound of formula E: 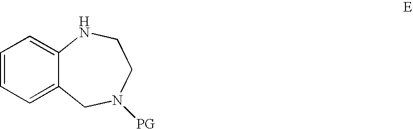
wherein, PG is a suitable amine protecting group,
(b) treating said compound of formula E with cyclopentene and paraformaldehyde, or an equivalent thereof, in the presence of a Lewis acid to give a compound of formula D: 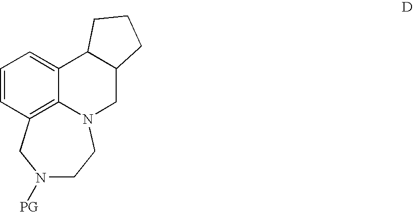
and, without isolation of said compound of formula D,
(c) treating said compound of formula D with hydrochloric acid to give amine salt C:

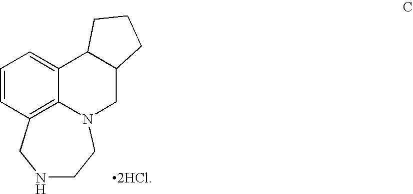

17. A compound of formula A-1:

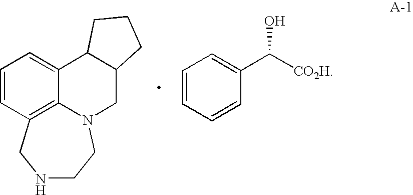

18. A compound: 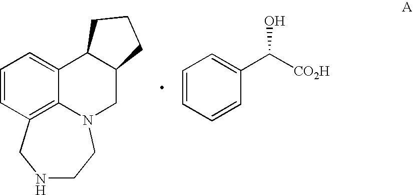
wherein said compound is diastereomerically enriched.

19. A compound: 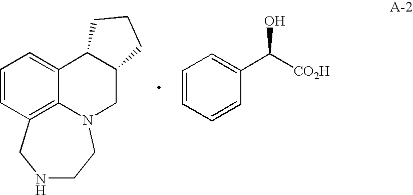
wherein said compound is diastereomerically enriched.

20. A method comprising the steps of:
(a) providing compound B: 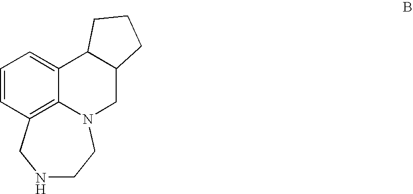
(b) treating said compound B with S-(+)-mandelic acid to form compound A-1: 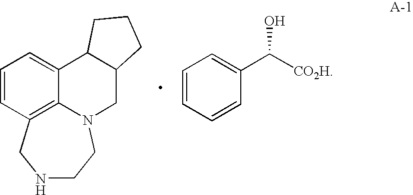
(c) obtaining compound A by suitable physical means: [chemical structure]
